(19) United States
(12) Patent Application Publication
(10) Pub. No.: US 2026/0306168 A1
(43) Pub. Date: Oct. 8, 2026

(54) ENERGY TREATMENT MEDICAL DEVICES, SYSTEMS, AND ENERGY TREATMENT METHODS

(71) Applicant: INTUITIVE SURGICAL OPERATIONS, INC., Sunnyvale, CA (US)

(72) Inventors: Alessandro Gozzi, Sunnyvale, CA (US); Ryan C. Abbott, San Jose, CA (US); Timothy O'Brien, Sunnyvale, CA (US); Harsukhdeep Singh Ratia, Sunnyvale, CA (US); Erasmo Lopez Calleros, Fremont, CA (US)

(21) Appl. No.: 19/638,606

(22) Filed: Apr. 3, 2026

Related U.S. Application Data
(60) Provisional application No. 63/783,515, filed on Apr. 4, 2025.
(60) Provisional application No. 63/783,543, filed on Apr. 4, 2025.

(51) Int. Cl. A61B 18/14; A61B 18/00

(52) U.S. Cl. CPC A61B 18/1492; A61B 2018/00083; A61B 2018/00577; A61B 2218/007

(57) ABSTRACT

Energy treatment systems and methods include a medical device with elongate bands having looped portions exposed distally, each of the elongate bands having electrodes extending longitudinally therealong. The systems and methods can include a drive assembly configured to move the bands relative to the housing to expand or retract the looped portions, and a control system operably coupled to the drive assembly.

### CROSS-REFERENCE TO RELATED APPLICATIONS

This application claims the benefit of priority to U.S. Provisional Application Nos. 63/783,515, filed Apr. 4, 2026, and 63/783,543, filed Apr. 4, 2026, the entireties of which are hereby incorporated by reference herein.

### FIELD

Disclosed embodiments relate to medical devices, and more specifically, to energy treatment medical devices.

### BACKGROUND

Minimally invasive medical techniques are intended to reduce the amount of tissue that is damaged during medical procedures, thereby reducing patient recovery time, discomfort, and harmful side effects. Such minimally invasive techniques may be performed through natural orifices in a patient anatomy or through one or more surgical incisions. Through these natural orifices or incisions, physicians may insert minimally invasive medical instruments (including surgical, diagnostic, therapeutic, and/or biopsy instruments) to reach a target tissue location. In minimally invasive robotically-assisted surgery, there is a time associated with switching out different instruments and/or end effectors. Reducing this time can advantageously reduce the time a patient is under general anesthesia.

### SUMMARY

The following presents a simplified summary of various examples described herein and is not intended to identify key or critical elements or to delineate the scope of the claims.

In accordance with a first example, an energy treatment medical device is disclosed that includes a housing, an elongate band having ends received within the housing and a looped intermediate portion disposed distally of the housing, and first and second electrodes spaced from one another along the elongate band, the first and second electrodes extending at least along the looped intermediate portion of the elongate band for energy treatment.

In some examples, the first and second electrodes are disposed in side-by-side relation and the medical device further includes an insulator extending along the elongate band separating the first and second electrodes and/or the second electrodes includes first and second portions disposed on either side of the first electrode, and the medical device includes insulators extending along the elongate band separating from the first electrode from the first and second portions of the second electrode.

In some examples, the medical device includes a leadscrew extending within the housing and a carriage rotatably coupled to the leadscrew and coupled to the housing, such that rotation of the leadscrew causes the carriage to translate longitudinally within the housing, the ends of the elongate band coupled to the carriage for deployment and retraction of the looped intermediate portion distally of the housing. In further examples, the medical device includes a motor received at least partially within the housing, the motor coupled to the leadscrew to drive rotation thereof for deployment and retraction of the looped intermediate portion of the elongate band; and/or includes an actuator interface comprising one or more capstans configured to be coupled to a backend mechanism, the one or more capstans coupled to the leadscrew to drive rotation thereof for deployment and retraction of the looped intermediate portion of the elongate band.

In some examples, the elongate band is a first elongate band and the medical device further includes a second elongate band having ends received within the housing and a looped intermediate portion disposed distally of the housing and first and second electrodes spaced from one another along the second elongate band, the first and second electrodes extending at least along the looped intermediate portion of the second elongate band for energy treatment. The first and second elongate bands have an intersecting end point and the looped intermediate portions are radially arrayed about a longitudinal axis to define a treatment area.

In some examples, the medical device includes a support post coupled to the looped intermediate portion of the elongate band, the support post extending proximally from the looped intermediate portion of the elongate band into the housing and movable to allow deployment and retraction of the elongate band. In further examples, the medical device includes a drive coupled to the support post, such that an insertion depth of the support post is adjustable to thereby adjust an amount of radial expansion of the elongate band, and/or the support post has a fixed insertion depth.

In some examples, the medical device includes a tissue engagement device disposed around the housing to engage tissue around an insertion opening to prevent withdrawal of the housing through the insertion opening.

In accordance with a second example, an energy treatment medical device is disclosed that includes a housing, a leadscrew received within the housing, a carriage rotatably coupled to the leadscrew and coupled with the housing, such that rotation of the leadscrew drives longitudinal movement of the carriage, and three elongate bands having electrodes extending longitudinally therealong, each elongate band having ends coupled to the carriage and a looped intermediate portion disposed distally of the housing, such that movement of the carriage causes expansion and retraction of the looped intermediate portions of the elongate bands.

In some examples, the electrodes include first and second electrodes spaced from one another along the elongate band, the first and second electrodes extending at least along the looped intermediate portion of the elongate band for energy treatment, and/or the electrodes include a first electrode and a second electrode including first and second portions disposed on either side of the first electrode.

In some examples, the elongate bands are connected together at an intersection point, the elongate bands being radially arrayed to provide expanded flexible strips or leaves defining a treatment area. In further examples, the medical device includes a support post coupled to the intersection point of the elongate bands, the support post extending proximally from the intersection point into the housing and being movable to allow deployment and retraction of the elongate bands.

In some examples, the intermediate looped portions of the elongate bands are deployable from and/or retractable into a storage position within the housing such that a diameter of looped intermediate portions distal of the housing is equal to or less than a diameter of the housing. In further examples, the medical device includes a motor at least partially received within the housing and coupled to the leadscrew to drive rotation of the leadscrew for deployment and retraction of the looped intermediate portions of the elongate bands.

Any of the above examples can further include one or more of the following aspects: the medical device includes an actuator interface comprising one or more capstans configured to be coupled to a backend mechanism, the one or more capstans coupled to the leadscrew to drive rotation thereof for deployment and retraction of the looped intermediate portions of the elongate bands; the leadscrew is configured to be manually rotated for deployment and retraction of the looped intermediate portions of the elongate bands; the housing defines an insufflation lumen configured to be coupled to an insufflation gas source to provide pressurized gas for insufflation at the treatment location and, optionally, the medical device includes a seal member extending around the housing, the seal member configured to engage tissue at an insertion opening to the treatment location; the housing defines a vacuum lumen configured to be coupled to a vacuum source for apposition at the treatment location prior to energy treatment; and/or the looped intermediate portions have an expansion ratio of at least 4.

In accordance with a third example, an energy treatment medical device is disclosed that includes a housing, a drive assembly, a carriage coupled to the drive assembly, operation of the drive assembly configured to drive longitudinal movement of the carriage, three elongate bands having electrodes extending longitudinally therealong, each elongate band having ends coupled to the carriage and a looped intermediate portion disposed distally of the housing, such that movement of the carriage causes expansion and retraction of the looped intermediate portions of the elongate bands, and a support shaft coupled to the drive assembly, the looped intermediate portions of the elongate bands coupled to an end of the support shaft, operation of the drive assembly configured to drive longitudinal movement of the support shaft to set an insertion depth for the elongate bands.

In some examples, the electrodes includes first and second electrodes spaced from one another along each of the elongate bands, the first and second electrodes extending at least along the looped intermediate portion of the elongate bands for energy treatment; and/or the electrodes include a first electrode and a second electrode including first and second portions disposed on either side of the first electrode.

In some examples, the drive assembly includes a first drive configured to drive longitudinal movement of the carriage and a second drive configured to drive longitudinal movement of the support shaft.

In further examples, the first drive includes a first leadscrew received within the housing and the carriage is rotatably coupled to the first leadscrew and coupled to the housing, such that rotation of the first leadscrew drives longitudinal movement of the carriage, and the second drive includes a second leadscrew received within the housing, and the support shaft is rotatably coupled to the second leadscrew and coupled to the housing, such that rotation of the second leadscrew drives longitudinal movement of the support shaft.

In yet further examples, the drive assembly includes a motor at least partially received within the housing and coupled to at least one of the first leadscrew or the second leadscrew to drive rotation thereof; the drive assembly includes an actuator interface including capstans configured to be coupled to a backend mechanism, the actuator interface coupled to the first leadscrew and the second leadscrew to drive rotation thereof; and/or at least one of the first leadscrew or the second leadscrew is configured to be manually rotated.

Any of the above examples can include one or more of the following aspects: the housing defines an insufflation lumen configured to be coupled to an insufflation gas source to provide pressurized gas for insufflation at a treatment location and/or the housing defines a vacuum lumen configured to be coupled to a vacuum source for apposition at a treatment location prior to energy treatment.

In accordance with a fourth example, an energy treatment medical device is disclosed that includes a housing, an elongate band having ends received within the housing and a looped intermediate portion disposed distally of the housing, the elongate band including electrodes extending at least along the looped intermediate portion of the elongate band for energy treatment, an insufflation lumen extending within the housing and configured to be coupled to an insufflation gas source to provide pressurized gas for insufflation at a treatment location, and a vacuum lumen extending within the housing and configured to be coupled to a vacuum source for apposition at a treatment location prior to energy treatment.

In some examples, the energy treatment medical device further includes a drive assembly configured to expand and retract the looped intermediate portion of the elongate band.

In some examples, the insufflation lumen and the vacuum lumen are defined by the housing and/or the elongate band includes a plurality of elongate bands.

In accordance with a fifth example, an energy treatment system is disclosed that includes an energy treatment medical device including a housing and elongate bands having looped portions exposed distally, each of the elongate bands having electrodes extending longitudinally therealong, a drive assembly configured to move the bands relative to the housing to expand or retract the looped portions, and a control system operably coupled to the drive assembly. The control system is configured to operate the drive assembly to expand the elongate bands at a treatment location and monitor operation of the drive assembly to determine when the elongate bands engage tissue during expansion.

In some examples, the control system is configured to monitor current draw of the drive assembly to determine when the elongate bands engage tissue during expansion, the system includes a user output and the control system is configured to notify a user of full expansion via the user output in response to determining that the elongate bands engaged tissue, and/or the control system is configured to stop operation of the drive assembly in response to determining that the elongate bands engaged tissue.

In some examples, the control system is configured to determine an expansion amount of the elongate bands based on the operation of the drive assembly. In further examples, the control system is configured to estimate a treatment area size based on the determined expansion amount and determine a rotation amount and number of rotations required to full treatment based on the treatment area size and a configuration of the electrodes on the elongate bands; and/or the control system is configured to determine one or more energy parameters associated with an energy treatment of the treatment area based on the determined expansion amount.

Any of the above examples can include one or more of the following aspects: the system includes an energy source coupled to the electrodes and the control system is configured to control the application of bipolar energy to the electrodes for an energy treatment; the system includes a roll drive and the control system is configured to operate the drive assembly to partially retract the elongate bands, operate the roll drive to rotate the energy treatment medical device to a second location, and operate the drive assembly to expand the elongate bands at the second treatment location; the drive assembly includes a leadscrew and motor, and the control system is configured to control operation of the motor to rotate the leadscrew; the housing defines an insufflation lumen configured to be coupled to an insufflation gas source and the control system is configured to provide pressurized gas through the insufflation lumen for insufflation at a treatment location; the housing defines a vacuum lumen configured to be coupled to a vacuum source and the control system is configured to control apply a vacuum through the vacuum lumen for apposition at a treatment location prior to an energy treatment; and/or the energy treatment medical device includes an insertion drive and a support post coupled between the insertion drive and the looped portions of the elongate bands, and the control system is configured to operate the insertion drive to set an insertion depth for the electrodes with the support post prior to operation of the drive assembly for expansion.

In accordance with a sixth example, an energy treatment method is disclosed that includes inserting an energy treatment medical device into a hollow sac or cyst, expanding arrayed elongate bands of the energy treatment medical device within the hollow sac or cyst to engage a first treatment area of an inner surface of the hollow sac or cyst with outer surfaces of the arrayed elongate bands, applying energy to electrodes extending along the outer surfaces of the arrayed elongate bands for an energy treatment, partially retracting the arrayed elongate bands of the energy treatment medical device to disengage from the arrayed elongate bands from the inner surface of the hollow sac or cyst, rotating the energy treatment medical device, expanding the arrayed elongate bands of the energy treatment medical device within the hollow sac or cyst to engage a second treatment area of the inner surface of the hollow sac or cyst with the outer surfaces of the arrayed elongate bands, applying energy to the electrodes for the energy treatment, repeating partial retraction, rotation, expansion, and application of energy to achieve full treatment coverage of the inner surface of the hollow sac or cyst, and fully retracting the arrayed elongate bands of the energy treatment medical device.

In some examples, the method includes insufflating the hollow sac or cyst to push tissue away from the arrayed elongate bands of the energy treatment medical device. In further examples, insufflating the hollow sac or cyst and partially retracting the arrayed elongate bands of the energy treatment medical device occurs simultaneously.

In some examples, the method includes cutting an access opening into the hollow sac or cyst and inserting the energy treatment medical device into the hollow sac or cyst includes inserting the energy treatment medical device through the access opening. In further examples, the method includes draining the hollow sac or cyst, the method includes engaging tissue around the access opening with a seal member after insertion of the energy treatment medical device to prevent withdrawal of the energy treatment medical device through the access opening, and/or the method includes pulling suction after expanding the arrayed elongate bands of the energy treatment medical device to pull the tissue against the electrodes extending along the outer surfaces of the arrayed elongate bands.

In some examples, the method includes monitoring operation of a drive assembly of the energy treatment medical device during expansion to determine when the arrayed elongate bands engage the tissue. In further examples, monitoring operation of the drive assembly includes monitoring a current draw of the drive assembly.

In some examples, the method includes determining an expansion amount of the arrayed elongate bands based on operation of a drive assembly of the energy treatment medical device and estimating a size of a treatment area of the inner surface of the hollow sac or cyst based on the determined expansion amount. In further examples, the method includes determining a rotation amount for full treatment coverage of the inner surface of the hollow sac or cyst based on the size of the treatment area and a configuration of the electrodes extending along the arrayed elongate bands; and/or the method includes determining one or more energy parameters associated with the energy treatment of the inner surface of the hollow sac or cyst based on determined expansion amount.

In some examples, expanding the arrayed elongate bands of the energy treatment medical device includes operating a motor to drive rotation of a leadscrew within the energy treatment medical device to drive longitudinal translation of a carriage rotatably coupled to the leadscrew, ends of the arrayed elongate bands being coupled to the carriage; and/or expanding the arrayed elongate bands of the energy treatment medical device includes spacing portions of the arrayed elongate bands to define a treatment volume having a diameter of at least 50 mm.

In accordance with a seventh example, an energy treatment method is disclosed that includes expanding the energy treatment medical device within tissue to engage a first treatment area with outer surfaces of arrayed elongate bands, applying energy to electrodes extending along the outer surfaces of the arrayed elongate bands for an energy treatment, insufflating the treatment location to push the tissue away from the arrayed elongate bands, and retracting the energy treatment medical device to disengage the arrayed elongate bands from the tissue.

In some examples, the tissue is a hollow sac or cyst and the method includes inserting an energy treatment medical device into the hollow sac or cyst.

In some examples, insufflating the treatment location and retracting the energy treatment medical device occurs simultaneously.

Any of the above examples can include one or more of the following aspects: the method includes rotating the energy treatment medical device after retraction, expanding the energy treatment medical device within the tissue to engage a second treatment area with the outer surfaces of the arrayed elongate bands, applying energy to the electrodes for the energy treatment, and repeating retraction, rotation, expansion, and application of energy to achieve full treatment coverage of the tissue; and/or the method includes pulling suction after expanding the energy treatment medical device to pull the tissue against the outer surfaces of the arrayed elongate bands.

It is to be understood that both the foregoing general description and the following detailed description are illustrative and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTIONS OF THE DRAWINGS

FIG. 1 is a schematic diagram for a robotically-assisted manipulator system, according to some examples.

FIG. 2A is a schematic diagram of an instrument system according to examples described herein.

FIG. 2B illustrates a distal portion of the instrument system of FIG. 2A with an extended example of an instrument according to examples described herein.

FIG. 3A is a perspective view of a medical device according to examples described herein.

FIG. 3B is a perspective view of a distal end portion of the medical device of FIG. 3A.

FIG. 4A is a perspective view of an energy treatment medical device according to examples described herein.

FIG. 4B is a top plan view of a first example elongate band for the energy treatment medical device of FIG. 4A.

FIG. 4C is a top plan view of a second example elongate band for the energy treatment medical device of FIG. 4A.

FIG. 4D is a sectional side view of the energy treatment medical device of FIG. 4A showing elongate bands in a retracted, storage position.

FIG. 4E is a sectional side view of the energy treatment medical device of FIG. 4A showing elongate bands in an expanded position.

FIG. 4F is a perspective view of a second example energy treatment medical device according to examples described herein.

FIG. 4G is a schematic diagram of a first medical system including the energy treatment medical device of FIG. 4A.

FIG. 4H is a schematic diagram of a second medical system including the energy treatment medical device of FIG. 4A.

FIG. 5A is a flowchart of a first portion of a method for energy treatment according to some examples described herein.

FIG. 5B is a flowchart of a second portion of the method for energy treatment of FIG. 5A.

Embodiments of the present disclosure and their advantages are best understood by referring to the detailed description that follows. It should be appreciated that like reference numerals are used to identify like elements illustrated in one or more of the figures, wherein showings therein are for purposes of illustrating embodiments of the present disclosure and not for purposes of limiting the same.

### DETAILED DESCRIPTION

In the following description, specific details are set forth describing some embodiments consistent with the present disclosure. Numerous specific details are set forth in order to provide a thorough understanding of the embodiments. It will be apparent, however, to one skilled in the art that some embodiments may be practiced without some or all of these specific details. The specific embodiments disclosed herein are meant to be illustrative but not limiting. One skilled in the art may realize other elements that, although not specifically described here, are within the scope and the spirit of this disclosure. In addition, to avoid unnecessary repetition, one or more features shown and described in association with one embodiment may be incorporated into other embodiments unless specifically described otherwise or if the one or more features would make an embodiment non-functional. In some instances, well known methods, procedures, components, and circuits have not been described in detail so as not to unnecessarily obscure aspects of the embodiments.

This disclosure describes various instruments and portions of instruments in terms of their state in three-dimensional space. As used herein, the term “position” refers to the location of an object or a portion of an object in a three-dimensional space (e.g., three degrees of translational freedom along Cartesian x-, y-, and z-coordinates). As used herein, the term “orientation” refers to the rotational placement of an object or a portion of an object (e.g., one or more degrees of rotational freedom such as, roll, pitch, and yaw). As used herein, the term “pose” refers to the position of an object or a portion of an object in at least one degree of translational freedom and to the orientation of that object or portion of the object in at least one degree of rotational freedom (e.g., up to six total degrees of freedom). As used herein, the term “shape” refers to a set of poses, positions, and/or orientations measured along an object. As used herein, the term “distal” refers to a position that is closer to a procedural site and the term “proximal” refers to a position that is further from the procedural site. Accordingly, the distal portion or distal end of an instrument is closer to a procedural site than a proximal portion or proximal end of the instrument when the instrument is being used as designed to perform a procedure.

Aspects of this disclosure herein can be part of a computer-assisted teleoperational manipulator system, sometimes referred to as a robotically-assisted manipulator system or a robotic system. The manipulator system can include one or more manipulators that can be operated with the assistance of an electronic controller (e.g., computer) to move and control functions of one or more instruments when coupled to the manipulators.

The systems, devices, and methods described herein utilize an energy treatment to treat tissue along an internal surface of a hollow space, sac, or organ. In some examples, the systems, devices, and methods can be utilized to treat ovarian and other types of cysts, as well as hollow sacs within the bladder. For example, the energy treatment device may be used to treat ovarian cysts while also minimizing damage to the ovarian reserve to provide easier recovery and reduce fertility complications.

In accordance with some examples herein, an opening into a hollow sac is created and, if necessary, the sac is drained of fluid. Flexible electrode bands are then inserted into the sac and expanded to fill an interior of the sac. Thereafter, bipolar energy is applied through the electrode bands to treat tissue along an interior of the sac. The electrodes bands can be retracted and rotated as needed to fully treat the interior surface of the sac (e.g., treating a desired portion or an entirety of the interior surface), avoiding the complexity and complications that can result from full removal of the sac, such as with a cystectomy, or inversion of the sac, such as with laser ovarian cyst ablation procedures.

In some examples, an energy treatment can include expanding an energy treatment medical device within a hollow space or sac (e.g., a cyst) to engage an interior surface of tissue with electrodes extending along arrayed elongate bands and applying energy to the electrodes for an energy treatment. The energy treatment further optionally includes partially retracting the energy treatment medical device to disengage from the tissue, rotating the energy treatment medical device, and reexpanding the energy treatment medical device to engage another portion of the interior surface of the tissue with the electrodes. These retraction, rotation, and reexpansion steps can be repeated as required to provide full coverage of an intended treatment area.

In some examples, a control system can control operation of the energy treatment medical device to provide an energy treatment. For example, the control system can operate a drive assembly of the energy treatment medical device to expand the elongate bands at a treatment location and monitor operation of the drive assembly to determine when the elongate bands engage tissue during expansion. Thereafter, the control system can estimate a size of the treatment area based on a determined expansion amount. With this estimate, the control system can further determine a rotation amount required for full coverage based on the treatment area size and an electrode configuration of the medical device and determine one or more energy parameters associated with energy treatment of the treatment area based on the determined expansion amount.

In some examples, an energy treatment medical device includes an elongate band having ends received within a housing and a looped intermediate portion disposed distally of the housing and two electrodes disposed in side-by-side relation on the elongate band. In further examples, the energy treatment medical device includes a leadscrew received within the housing and a carriage rotatably coupled to the leadscrew and coupled with the housing. The elongate bands have ends coupled to the carriage, such that movement of the carriage due to rotation of the leadscrew causes expansion and retraction of the looped intermediate portions of the elongate bands. In some examples, the electrodes are disposed on outer surfaces of the elongate bands, such that expansion of the bands causes the outer surfaces of the bands to engage tissue for energy treatment. In other examples, the electrodes are disposed on inner surfaces of the elongate bands, such that retraction of the bands causes the inner surfaces of the bands to engage tissue for energy treatment. In other examples, the electrodes are disposed on both the inner surfaces and the outer surfaces of the elongate bands. In some examples where electrodes are disposed on the inner and outer surfaces, the electrodes can be selectively activated or activated at different times (e.g., only the inner surface electrodes are activated, only the outer surfaces electrodes are activated, or both the inner and outer surface electrodes are activated).

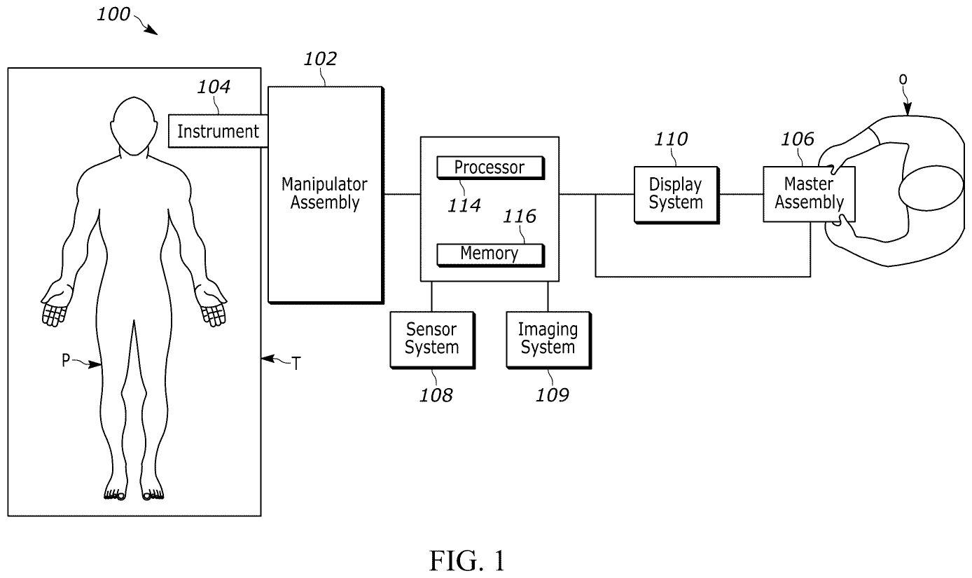

FIG. 1 illustrates an embodiment of a robotically-assisted manipulator system for use with the tools described herein. The manipulator system can be used, for example, in surgical, diagnostic, therapeutic, biopsy, or non-medical procedures, and is generally indicated by the reference numeral 100. As shown in FIG. 1, a robotically-assisted manipulator system 100 can include one or more manipulator assemblies 102 for operating one or more medical instrument systems 104 in performing various procedures on a patient P positioned on a table T in a medical environment 101. For example, the manipulator assembly 102 can drive catheter or end effector motion, can apply treatment to target tissue, and/or can manipulate control members. The manipulator assembly 102 can be teleoperated, non-teleoperated, or a hybrid teleoperated and non-teleoperated assembly with select degrees of freedom of motion that can be motorized and/or teleoperated and select degrees of freedom of motion that can be non-motorized and/or non-teleoperated. An operator input system 106, which can be inside or outside of the medical environment 101, generally includes one or more control devices for controlling manipulator assembly 102. Manipulator assembly 102 supports medical instrument system 104 and can optionally include a plurality of actuators or motors that drive inputs on medical instrument system 104 in response to commands from a control system 112. The actuators can optionally include drive systems that when coupled to medical instrument system 104 can advance medical instrument system 104 into a naturally or surgically created anatomic orifice. Other drive systems can move the distal end of medical instrument in multiple degrees of freedom, which can include three degrees of linear motion (e.g., linear motion along the X, Y, Z Cartesian axes) and in three degrees of rotational motion (e.g., rotation about the X, Y, Z Cartesian axes). The manipulator assembly 102 can support various other systems for irrigation, treatment, or other purposes. Such systems can include fluid systems (including, for example, reservoirs, heating/cooling elements, pumps, and valves), generators, lasers, interrogators, and ablation components.

Robotically-assisted manipulator system 100 also includes a display system 110 for displaying an image or representation of the surgical site and medical instrument system 104 generated by an imaging system 109 which can include an imaging system, such as an endoscopic imaging system. Display system 110 and operator input system 106 can be oriented so an operator O can control medical instrument system 104 and operator input system 106 with the perception of telepresence. A graphical user interface can be displayable on the display system 110 and/or a display system of an independent planning workstation.

In some examples, the endoscopic imaging system components of the imaging system 109 can be integrally or removably coupled to medical instrument system 104. However, in some examples, a separate imaging device, such as an endoscope, attached to a separate manipulator assembly can be used with medical instrument system 104 to image the surgical site. The endoscopic imaging system 109 can be implemented as hardware, firmware, software, or a combination thereof which interact with or are otherwise executed by one or more computer processors, which can include the processors of the control system 112.

Robotically-assisted manipulator system 100 can also include a sensor system 108. The sensor system 108 can include a position/location sensor system (e.g., an actuator encoder or an electromagnetic (EM) sensor system) and/or a shape sensor system (e.g., an optical fiber shape sensor) for determining the position, orientation, speed, velocity, pose, and/or shape of the medical instrument system 104. The sensor system 108 can also include temperature, pressure, force, or contact sensors or the like.

Robotically-assisted manipulator system 100 can also include a control system 112. Control system 112 includes at least one memory 116 and at least one computer processor 114 for effecting control between medical instrument system 104, operator input system 106, sensor system 108, and display system 110. Control system 112 also includes programmed instructions (e.g., a non-transitory machine-readable medium storing the instructions) to implement a procedure using the robotically-assisted manipulator system including for navigation, steering, imaging, engagement feature deployment or retraction, applying treatment to target tissue (e.g., via the application of energy), or the like.

Control system 112 can optionally further include a virtual visualization system to provide navigation assistance to operator O when controlling medical instrument system 104 during an image-guided surgical procedure. Virtual navigation using the virtual visualization system can be based upon reference to an acquired pre-operative or intra-operative dataset of anatomic passageways. The virtual visualization system processes images of the surgical site imaged using imaging technology such as computerized tomography (CT), magnetic resonance imaging (MRI), fluoroscopy, thermography, ultrasound, optical coherence tomography (OCT), thermal imaging, impedance imaging, laser imaging, nanotube X-ray imaging, and/or the like. The control system 112 can use a pre-operative image to locate the target tissue (using vision imaging techniques and/or by receiving user input) and create a pre-operative plan, including an optimal first location for performing treatment. The pre-operative plan can include, for example, a planned size to expand an expandable device, a treatment duration, a treatment temperature, and/or multiple deployment locations.

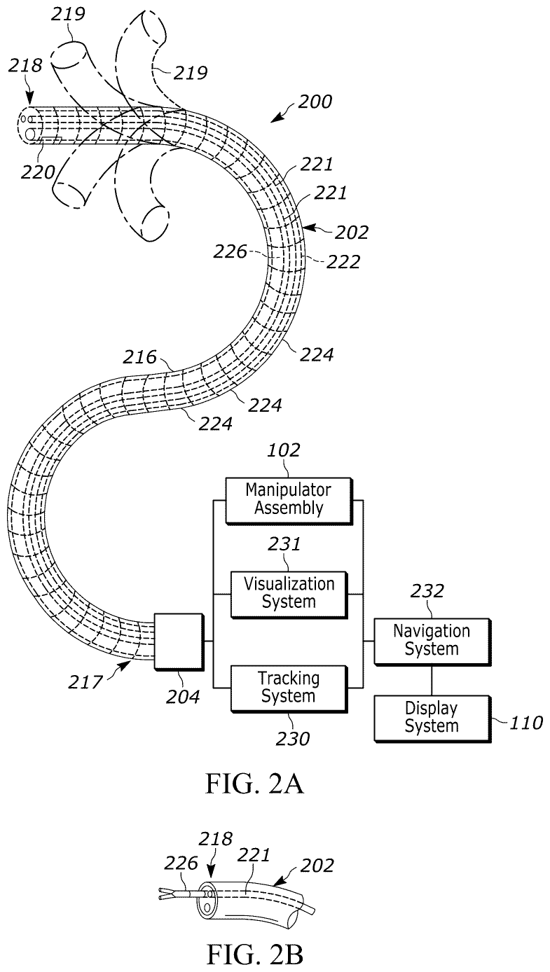

FIG. 2A shows a medical instrument system 200 according to some embodiments. In some embodiments, medical instrument system 200 can be used in an image-guided medical procedure. In some examples, medical instrument system 200 can be used for non-teleoperational exploratory procedures or in procedures involving traditional manually operated medical instruments, such as endoscopy. In some embodiments, medical instrument system 200 is interchangeable with, or a variation of, medical instrument system 104 of FIG. 1.

Medical instrument system 200 includes elongate flexible device 202, such as a flexible catheter or endoscope (e.g., gastroscope, bronchoscope), coupled to a drive unit 204. Elongate flexible device 202 includes a flexible body 216 having proximal end 217 and distal end, or tip portion, 218. In some embodiments, flexible body 216 has an approximately 14-20 mm outer diameter. Other flexible body outer diameters can be larger or smaller. Flexible body 216 can have an appropriate length to reach certain portions of the anatomy, such as the lungs, sinuses, throat, or the upper or lower gastrointestional region, when flexible body 216 is inserted into a patient's oral or nasal cavity.

Flexible body 216 includes one or more channels 221 sized and shaped to receive one or more medical instruments 226. In some embodiments, flexible body 216 includes two channels 221 for separate instruments 226, however, a different number of channels 221 can be provided. FIG. 2B is a simplified diagram of flexible body 216 with medical instrument 226 extended according to some embodiments. In some embodiments, medical instrument 226 can be used for procedures and aspects of procedures, such as surgery, biopsy, ablation, mapping, imaging, illumination, irrigation, or suction. Medical instrument 226 can be deployed through channel 221 of flexible body 216 and used at a target location within the anatomy. Medical instrument 226 can include, for example, image capture devices, biopsy instruments, ablation instruments, catheters, laser ablation fibers, and/or other surgical, diagnostic, or therapeutic tools. Medical tools can include end effectors having a single working member such as a scalpel, a blunt blade, a lens, an optical fiber, an electrode, and/or the like. Other end effectors can include, for example, forceps, graspers, balloons, needles, scissors, clip appliers, and/or the like. Other end effectors can further include electrically activated end effectors such as electrosurgical electrodes, transducers, sensors, imaging devices and/or the like. Medical instrument 226 can be advanced from the opening of channel 221 to perform the procedure and then retracted back into the channel when the procedure is complete. Medical instrument 226 can be removed from proximal end 217 of flexible body 216 or from another optional instrument port (not shown) along flexible body 216. The medical instrument 226 can be used with an image capture device (e.g., an endoscopic camera) also within the elongate flexible device 202. Alternatively, the medical instrument 226 can itself be the image capture device.

Medical instrument 226 can additionally house cables, linkages, or other actuation controls (not shown) that extend between its proximal and distal ends to controllably the bend distal end of medical instrument 226. Flexible body 216 can also house cables, linkages, or other steering controls (not shown) that extend between drive unit 204 and distal end 218 to controllably bend distal end 218 as shown, for example, by broken dashed line depictions 219 of distal end 218. In some embodiments, medical instrument system 200 can include gripping features, manual actuators, or other components for manually controlling the motion of medical instrument system 200.

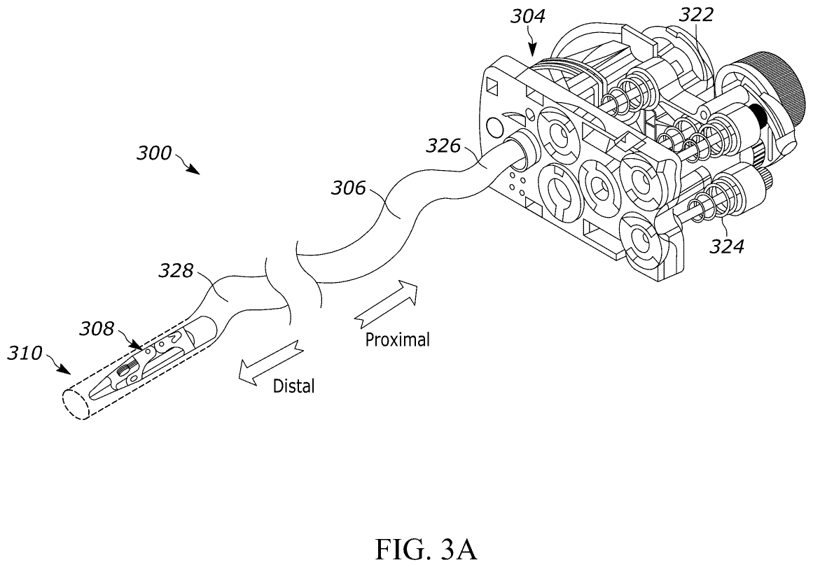

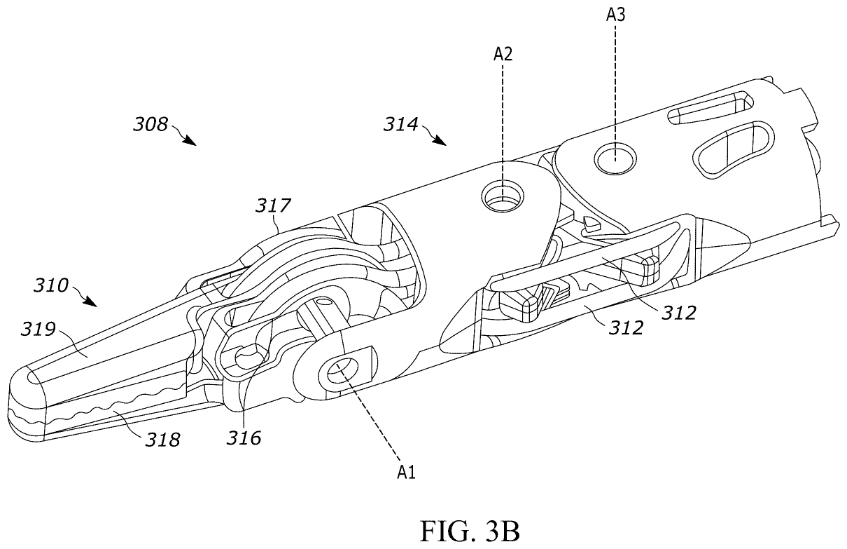

FIGS. 3A and 3B are various views of a medical device 300, according to an embodiment. In some embodiments, the medical device 300 or any of the components therein are optionally parts of an instrument for a surgical system that performs surgical procedures, and which surgical system can include a manipulator unit, a series of kinematic linkages, a series of cannulas, or the like. The medical device 300 (and any of the instruments described herein) can be used in any suitable surgical system, such as the manipulator system 100 or the manipulator system 200 shown and described above. The medical device 300 can be used as the medical instruments 226 described above. As shown in FIG. 3A, the medical device 300 defines (or is included within) a distal boundary (or footprint) 302 that corresponds to a cannula size, or a size to fit within a working channel of an elongate flexible device (such as a flexible catheter or endoscope), or other size dictated by the surgical environment. The distal boundary 302 can be a cylindrical shape having any suitable nominal diameter (e.g., 8 mm, 5 mm or any size therebetween). The medical device 300 includes a force transmission mechanism 304, a shaft 306, an optional distal wrist assembly 308, a distal end effector 310, and a set of tension elements 312 (which can be, for example, a cable, band, or the like).

The medical device 300 can include multiple tension elements 312. For example, in some embodiments, the medical device 300 can include two tension elements 312 with each tension element 312 having two segments extending along the shaft 306 of the instrument, thereby forming four proximal end portions. Referring to FIG. 3B, respective tension elements 312 can be routed through a wrist assembly 314 and wrapped about respective pulleys 316 or 317 of the tool members 318, 319. Each tension element 312 has two tension element segments along the shaft 306 with two proximal end portions that, when moved in opposite directions, can (among other things) cause rotation of the respective tool member 318 or 319 about the axis A1. The tension elements 312 can be, for example, cables, bands, or the like that couple the force transmission mechanism 304 to the distal wrist assembly 308 and end effector 310. In some embodiments, the tension elements 312 can be constructed from a polymer.

The medical device 300 is configured such that movement of one or more of the tension elements 312 produces rotation of the end effector 310 about a first rotation axis A1 (see FIG. 3B, which functions as a yaw axis, the term yaw is arbitrary), rotation of the wrist assembly 308 about a second rotation axis A2 and/or optionally about a third rotation axis A3 (which functions as a pitch axis, the term pitch is arbitrary), a cutting rotation of the tool members of the end effector 310 about the first rotation axis A1, or any combination of these movements. Changing the pitch or yaw of the medical device 300 can be performed by manipulating the tension elements 312 in a similar manner as that described with reference to the device 2400 described in copending International Patent Application Serial No. PCT/US2022/039942, entitled “Surgical Instrument Cable Control and Routing Structures,” the disclosure of which is incorporated herein by reference in its entirety.

As shown in FIG. 3A, the proximal force transmission mechanism 304 includes a set of drive components such as capstans 322 and 324 that rotate or “wind” a proximal portion of any of the tension elements 312 to produce the desired tension element movement. In some embodiments, two proximal ends of a tension element 312, which are associated with opposing directions of a single degree of freedom, are connected to two independent drive capstans 322 and 324. This arrangement, which is generally referred to as an antagonist drive system, allows for independent control of the movement of (e.g., pulling in or paying out) each of the ends of the tension elements 312. The force transmission mechanism 304 produces movement of the tension elements 312, which operates to produce the desired articulation movements (pitch, yaw, or grip) at the wrist assembly 308 and end effector 310.

Moreover, although the force transmission mechanism 304 is shown as including capstans, in other embodiments, a force transmission mechanism can include one or more linear actuators that produce translation (linear motion) of a portion of the cables. Such force transmission mechanisms can include, for example, a gimbal, a lever, or any other suitable mechanism to directly pull (or release) an end portion of any of the cables. For example, in some embodiments, the proximal force transmission mechanism 304 can include any of the proximal force transmission mechanisms or components described in U.S. Patent Application Pub. No. US 2015/0047454 A1 (filed Aug. 15, 2014), entitled “Lever Actuated Gimbal Plate,” or U.S. Pat. No. 6,817,974 B2 (filed Jun. 28, 2001), entitled “Surgical Tool Having Positively Positionable Tendon-Actuated Multi-Disk Wrist Joint,” each of which is incorporated herein by reference in its entirety.

The shaft 306 can be any suitable elongated shaft that is coupled to the force transmission mechanism 304 and to the optional wrist assembly 308 (when present) or the end effector 310. Specifically, the shaft 306 includes a proximal portion 326 that is coupled to the force transmission mechanism 304, and a distal portion 328 that is coupled to the optional wrist assembly 308 or to the end effector 310. The shaft 306 defines a passageway or series of passageways through which the tension elements 312 and other components (e.g., electrical wires, ground wires, or the like) can be routed from the force transmission mechanism 304 to the wrist assembly 308. In some embodiments, the shaft 306 can be a substantially rigid member, while in other embodiments, the shaft 306 can be a flexible member.

Other configurations of teleoperated manipulator systems are also contemplated, such as systems configured for multi-port or single-port procedures. For example, the embodiments described herein can be used with a da Vinci® Surgical System, such as the da Vinci X®, Xi®, or SP® Surgical Systems, all commercialized by Intuitive Surgical, Inc., of Sunnyvale, California.

In these examples, one or more instruments includes shafts having at a distal end a moveable end effector or an endoscope, camera, or other sensing device, and can or cannot include a wrist mechanism (not shown) to control the movement of the distal end. In these examples, the shafts can be partially or entirely rigid (e.g., not bendable/articulable). As such, these instruments can be utilized through the cannula to directly access a treatment location, relying on actuation of end effector mechanisms to perform desired operations during a procedure.

In some approaches, the distal end portions of the instruments of these examples are received through a port structure (e.g., single or multi-port structure) to be introduced into the patient. Suitable port structures can include a cannula and an instrument entry guide inserted into the cannula. Individual instruments are inserted into the entry guide to reach a surgical site.

The manipulator systems described herein are not limited to the embodiments of FIGS. 1-3B, and various other teleoperated, computer-assisted manipulator configurations can be used with the embodiments described herein. The diameter or diameters of an instrument shaft and end effector are generally selected according to the size of the cannula with which the instrument will be used and depending on the surgical procedures being performed.

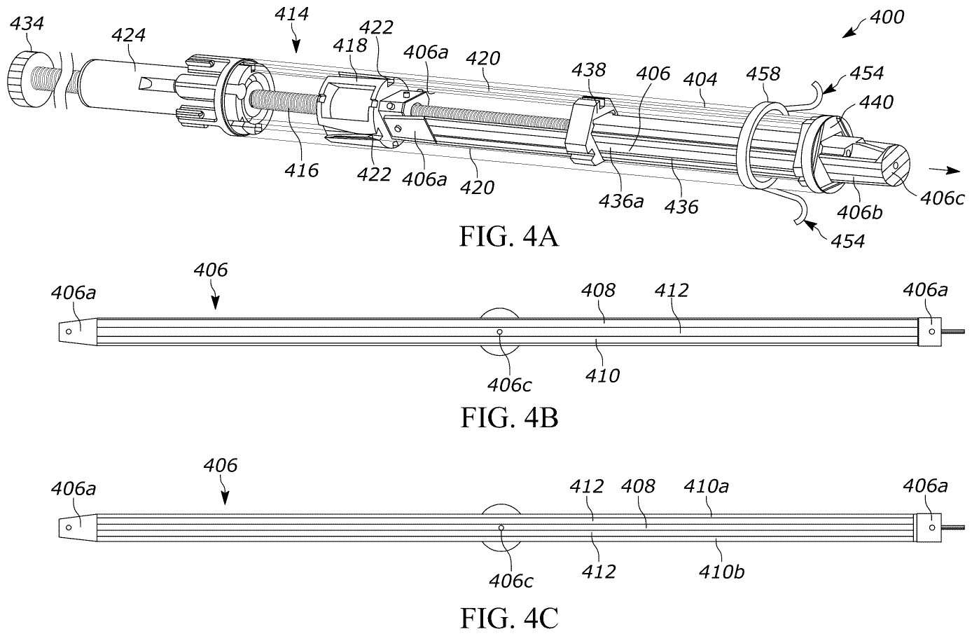

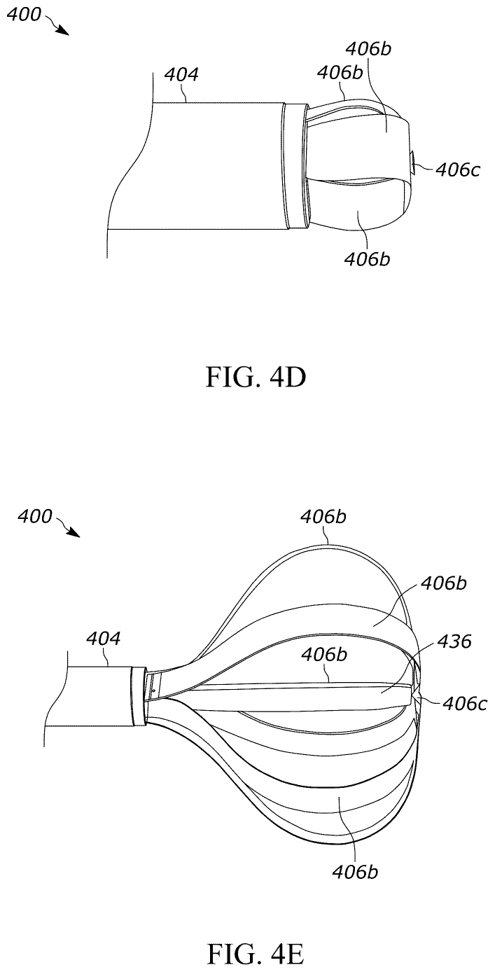

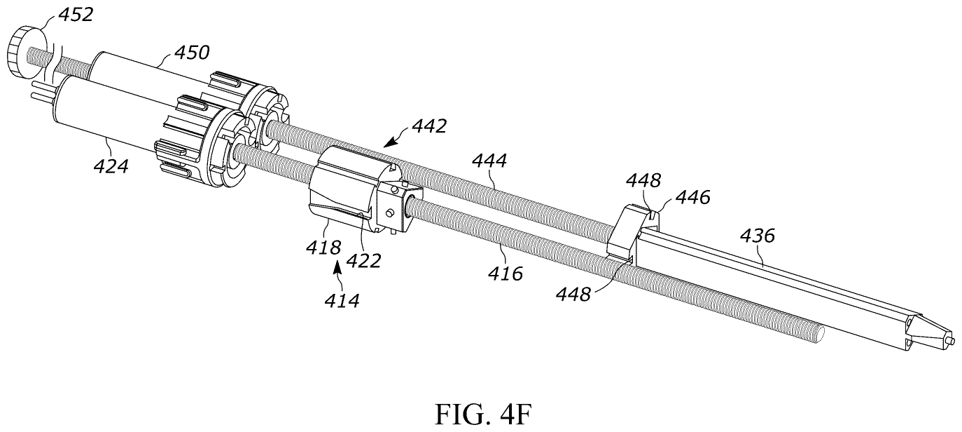

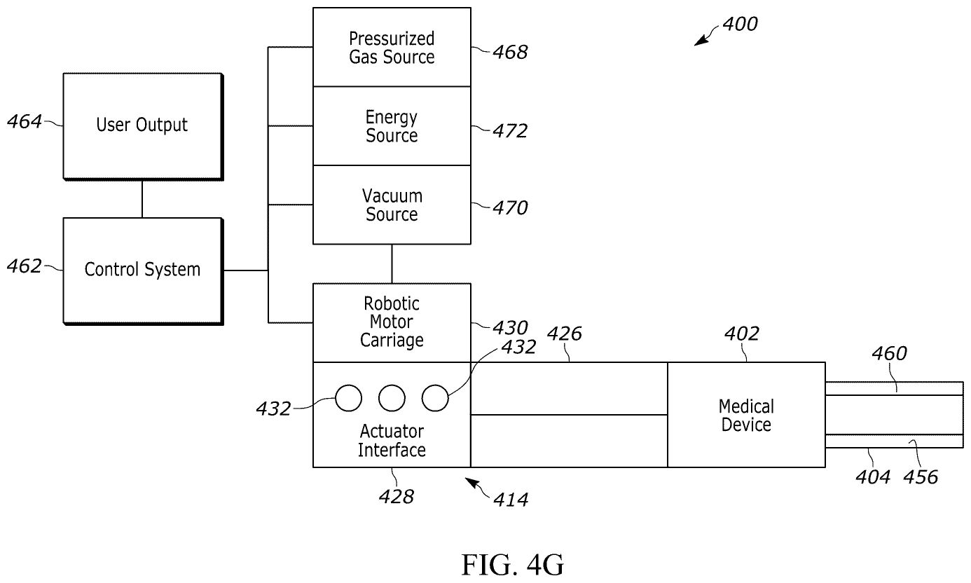

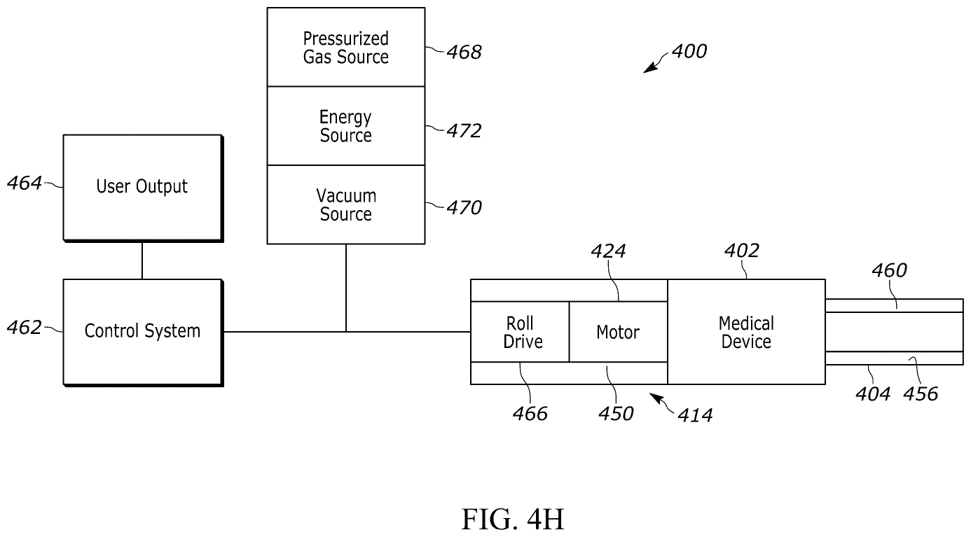

FIGS. 4A-4H show a medical system 400 and components thereof according to some embodiments. The medical system 400 includes a medical device 402, such as an end effector, capable of performing surface energy treatment. The energy treatment can include a single dose or can include a plurality of predetermined doses.

The medical device 402 includes a housing 404 and one or more elongate bands 406 having ends 406a received within the housing 404 and a looped intermediate portion 406b disposed distally of the housing 404. The housing 404 can extend along a longitudinal axis L. As shown, a first electrode 408 and a second electrode 410 are spaced from one another along the elongate band 406 and extend at least along the looped intermediate portion 406b of the elongate band 406 for energy treatment.

The electrodes 408, 410 can have any suitable configuration. In a first example as shown in FIG. 4B, the first and second electrodes 408, 410 each have a single portion (e.g., single conductive strip), such that the first and second electrodes 408, 410 are disposed in side-by-side relation with an insulator 412 extending along the elongate band 406 separating the single conductive strips of the first and second electrodes 408, 410. In a second example as shown in FIG. 4C, the second electrode 410 comprises first and second portions 410a, 410b disposed on either side of the first electrode 408 with insulators 412 extending along the elongate band 406 separating the first electrode 408 from the first and second portions 410a, 410b of the second electrode 410. As described herein, the elongate bands 406 can be configured with the electrodes 408, 410 exposed along an outer surface, such that the bands 406 are expanded into tissue for an energy treatment or can be configured with the electrodes 408, 410 exposed along an inner surface, such that the bands are retracted onto tissue for an energy treatment.

The electrodes 408, 410 can be disposed along an outer surface of the elongate band 406, such that an energy treatment is performed on the exterior of the looped intermediate portions 406b and the band 406 can be expanded to contact tissue at the outer surface of the band 406 for treatment. Alternatively, the electrodes 408, 410 can be disposed along an inner surface of the elongate band 406, such that an energy treatment is performed on the interior of the looped intermediate portions 406b and the band 406 can be retracted to contact tissue at the inner surface of the band 406 for treatment. In other examples, the electrodes are disposed on both the inner surfaces and the outer surfaces of the elongate band 406. In some examples where electrodes are disposed on the inner and outer surfaces, the electrodes can be selectively activated or activated at different times (e.g., only the inner surface electrodes are activated, only the outer surfaces electrodes are activated, or both the inner and outer surface electrodes are activated).

In some examples, the medical device 402 includes a single elongate band 406. In other examples, the medical device 402 includes a plurality of elongate bands 406 each with ends 406a received within the housing 404 and a looped intermediate portion 406b disposed distally of the housing 404, such as two elongate bands 406, three elongate bands 406, or more. The elongate bands 406 can have the same configuration for the first and second electrodes 408, 410, or can have differing configurations.

The looped intermediate portions 406b of the elongate bands 406 have an intersecting end point 406c with the elongate bands 406 overlapping one another. As shown, the intersecting end point 406c can be aligned along the longitudinal axis L of the housing 404. The looped intermediate portions 406b are radially arrayed about the longitudinal axis L to define a volume (e.g., generally spherical volume) therebetween and a treatment area. The radially arrayed looped intermediate portions 406b provide expanded flexible strips or leaves on either side of the intersecting end point 406c to define the treatment area.

In some examples, the intermediate looped portions 406b of the elongate bands 406 are deployable from and/or retractable into a storage position within the housing 404. As shown in FIG. 4D, a majority of the elongate bands 406 are received within the housing 404 in the storage position, with the intersecting end point 406c disposed just distal of the housing 404. In some embodiments, a portion or an entirety of the looped portions 406b may be retractable into the housing 404. By retracting the bands 406 to the storage position, a diameter of the looped intermediate portions 406b distal of the housing 404 can optionally be equal to or less than a diameter of the housing 404. This configuration allows the medical device 402 to be delivered to a treatment location through a lumen or other structure having a diameter corresponding to the housing 404. Thereafter, as discussed in more detail below, the looped intermediate portion 406b of the elongate bands 406 can be deployed to a treatment position with an expanded diameter. It will be understood that the treatment position for a given procedure may not correspond with a maximum possible expansion diameter. Instead, the medical device 402 can be configured to expand to a size corresponding to a treatment location, as described in more detail below.

In some examples, the looped intermediate portions have a maximum expansion ratio between the storage position and the treatment position of at least 4, at least 5, at least 6, or at least 6.5. In some examples, the diameter of the intermediate looped portions 406 b in the storage position is about 12 mm and about 50 mm in a maximum treatment position, about 70 mm in a maximum treatment position, or about 80 mm in a maximum treatment position.

An example drive system 414 for deployment and retraction of the elongate bands 406 is shown in FIG. 4A. The drive system 414 includes a leadscrew 416 extending within the housing 404 and a carriage 418 rotatably coupled to the leadscrew 416. As shown, the carriage 418 is coupled to the housing 404, such as by a splined connection. In some examples, the housing 404 includes one or more longitudinal ribs 420 and the carriage 418 includes openings 422 to receive the ribs 420 therethough. Accordingly, engagement with the ribs 420 prevents the carriage 418 from rotating relative to the housing 404 as the leadscrew 416 is actuated, and instead drives longitudinal movement of the carriage 418 as the leadscrew 416 is actuated. In another example, the carriage 418 can include ribs that engage openings/channels defined in the housing 404.

The ends 406a of the elongate band(s) 406 are coupled to the carriage 418, such that longitudinal movement of the carriage 418 causes the looped intermediate portion(s) 406b to be selectively deployed to the treatment position (via distal movement of the carriage 418) and selectively retracted to the storage position (via proximal movement of the carriage 418). In one example, the ends 406a of the elongate bands 406 and carriage 418 are mechanically coupled via one or more protrusions and openings as shown. For example, the ends 406a of the bands 406 or the carriage 418 may have one or more protrusions, and the other of the ends 406a or carriage 418 may have openings to receive the protrusions and form mechanical connections. In other examples, the ends 406a can be adhered, welded, frictionally engaged, or otherwise affixed to the carriage 418.

Rotation of the leadscrew 416 can be achieved by any suitable mechanism. In one example, the medical device 402 include an integrated, on-board motor 424 at least partially received within the housing 404, such that the medical device 402 is a motorized laparoscopic instrument. With this configuration, the motor 424 is coupled to the leadscrew 416 within the housing 404 and selectively drives the rotation of the leadscrew 416 to deploy or retract the elongate bands 406. The medical device 402 of this example can be disposed on the end of a flexible or rigid elongate instrument or can be a separate device configured to be grasped by a separate instrument.

In other examples, the medical device 402 is an end effector of a robotic instrument 426. For example, the robotic instrument 426 can include an integrated back-end motor 424 or the robotic instrument can include a force transmission mechanism 428 to receive actuation via a robotic motor carriage 430 separate from the medical device 402. As shown in FIG. 4G, the force transmission mechanism 428 can include one or more robotic inputs 432 configured to be coupled to outputs of the robotic motor carriage 430 or other backend mechanism. The force transmission mechanism 428 includes a set of drive components such as capstans, pulleys, and/or tension elements that are coupled to the drive system 414 and transfer forces/torques from the robotic motor carriage 430 to the drive system 414. For example, one or more capstans in the force transmission mechanism 428 may be operatively coupled to the leadscrew 416 to drive rotation thereof for deployment and retraction of the elongate band(s) 406.

In additional or alternative examples, the medical device 402 can include an exposed user input control 434 that allows a user to manually rotate the leadscrew 416 for deployment and retraction of the elongate band(s) 406. The control 434 can be a wheel or knob coupled directly to the leadscrew or via a linkage.

Although expansion and retraction of the looped intermediate portion(s) 406b of the elongate band(s) 406 can be operated without additional structure as discussed, as shown in FIGS. 4A and 4E, the medical device 402 can further include a support post 436 that is coupled to the looped intermediate portion(s) 406b the elongate band(s) 406, such as to the intersecting end point 406c. The support post 436 extends proximally from the band(s) 406 into the housing 404 and includes a proximal end 436a movably mounted within the housing 404, such that the support post 436 shifts into and out of the housing 404 to allow deployment and retraction of the elongate band(s) 406.

In some examples, the proximal end 436a of the support post 436 freely floats within the housing 404, such that the support post 436 is only connected (e.g., secured) to the elongate band(s) 406. With this configuration, the connection with the support post 436 holds the elongate bands 406 together and restricts lateral movement of the elongate bands 406 due to the engagement of the support post 436 with the housing 404. In other examples, the proximal end 436a can include an outwardly extending flange 438 that is configured to abut a rim or cap 440 extending around the distal opening of the housing 404 when the support post 436 is driven fully out of the housing 404. For example, as the looped intermediate portion(s) 406b of the elongate band(s) 406 are driven out of the housing 404, the support post 436 is pulled along with the expansion of the band(s) 406. Eventually, based on the size of the elongate band(s) 406 and the longitudinal length of the support post 436, the flange 438 is pulled into engagement with the rim 440 to stop further longitudinal movement of the support post 436 relative to the housing 404. With this configuration, the longitudinal length of the support post 436 can be utilized to restrict or set an insertion depth of the looped intermediate portions 406b of the elongate bands 406, which also sets the available radial expansion of the looped intermated portions 406b.

In other examples, the medical device 402 can include a drive system 442 for insertion and withdrawal of the support post 436. An example drive system 442 is shown in FIG. 4F. The drive system 442 includes a leadscrew 444 extending within the housing 404 and a carriage 446 rotatably coupled to the leadscrew 444. As shown, the carriage 446 is coupled to the housing 404, such as by a splined connection. In some examples, the housing 404 includes one or more longitudinal ribs 420 and the carriage 446 includes openings 448 to receive the ribs 420 therethough. Accordingly, engagement with the ribs 420 prevents the carriage 446 from rotating relative to the housing 404 as the leadscrew 444 is actuated, and instead drives longitudinal movement of the carriage 446 as the leadscrew 444 is actuated. The proximal end 436a of the support post 436 is coupled to the carriage 446, such that movement of the carriage 446 longitudinally causes the support post 436 to be inserted distally of the housing 404 and withdrawn into the housing 404. In another example, the carriage 446 can include ribs that engage openings/channels defined in the housing 404.

In some examples, the carriages 446, 418 are rotationally aligned/rotationally fixed relative to the housing 404 using common alignment features (e.g., ribs and/or openings/channels) of the housing 404. In other examples, the carriages 446, 418 can be rotationally aligned/rotationally fixed relative to the housing 404 by engagement with separate alignment features (e.g., ribs and/or openings/channels) of the housing 404 dedicated for each carriage 446, 418.

Rotation of the leadscrew 444 can be achieved by any suitable mechanism. In one example, the medical device 402 include an integrated, on-board motor 450 at least partially received within the housing 404. With this configuration, the motor 450 is coupled to the leadscrew 444 within the housing 404 and selectively drives the rotation of the leadscrew 444 to insert or withdraw the support post 436. The medical device 402 of this example can be disposed on the end of a flexible or rigid elongate instrument or can be a separate device configured to be grasped by a separate instrument.

In other examples, the medical device 402 is an end effector of a robotic instrument 426. For example, the robotic instrument 426 can include an integrated back-end motor 450 or the robotic instrument can include the actuator interface 428 to receive actuation via the robotic motor carriage 430. As shown, one or more of the capstans 432 of the actuator interface 428 can be configured to be coupled to the robotic motor carriage 430 or other backend mechanism. The capstans 432 of this example are coupled to the leadscrew 444 to drive rotation thereof for insertion and withdrawal of the support post 436.

In additional or alternative examples, the medical device 402 can include an exposed control 452 that allows a user to manually rotate the leadscrew 444 for insertion and withdrawal of the support post 436. The control 452 can be a wheel or knob coupled directly to the leadscrew or via a linkage.

With the drive system 442 of any of the above examples, an insertion depth of the support post 436 is adjustable to thereby adjust an amount of radial expansion of the elongate band(s) 406. In some examples, the drive system 442 for the support post 436 is operable to be driven independently of the drive system 414 for the elongate band(s) 406. In various embodiments, the drive systems 414 and 442 can be controlled separately or in concert to control an amount of expansion diameter of the elongate band(s) 406. For example, with the same deployment amount of the elongate band(s) 406, positioning the support post 436 in a relatively shorter distal deployment results in a greater maximum radial width of the looped intermediate portions 406b, as opposed to a relatively lesser maximum radial width of the looped intermediate portions 406b by positioning the support post 436 in a relatively longer distal deployment.

According to one example use, the medical device 402 can be utilized to treat tissue along an interior surface of a cyst, hollow space, sac, or organ. By way of example for a cyst, the medical device 402 can be inserted through an opening cut into the cyst with the elongate bands 406 in the storage position and subsequently expanded to engage the interior surface of the cyst. In some examples, the medical device 402 includes a tissue engagement device 454 disposed around the housing 404 to engage tissue around the insertion opening to prevent withdrawal of the housing 404 through the insertion opening. The tissue engagement device 454 can be any suitable structure or mechanism, such as one or more splines, hooks, protrusions, ribs, o-rings, and so forth.

In some examples, a treatment procedure may benefit from insufflation. Accordingly, in some examples and as shown in FIGS. 4G and 4H, the medical device 402 optionally includes an insufflation lumen 456 extending longitudinally therealong with a delivery opening at the distal end of the housing 404. The insufflation lumen 456 can have any suitable configuration, such as being the main lumen/cavity of the housing 404, a separately defined lumen integrated into the housing 404, or a separate tube extending through the housing 404. The insufflation lumen 456 is configured to be fluidly coupled to a suitable insufflation gas source (e.g., CO2) to provide pressurized gas for insufflation at the treatment location. If desired, the medical device 402 may also include a seal member 458 extending around the housing 404, where the seal member 458 is configured to engage tissue at the insertion opening to the treatment location.

In some examples, a treatment procedure may benefit from apposition at the treatment location and as shown in FIGS. 4G and 4H, such as prior to an energy treatment to pull tissue against the electrodes 408, 410. Accordingly, in these examples, the medical device 402 includes a vacuum lumen 460 extending longitudinally therealong with a suction opening at the distal end of the housing 404. The vacuum lumen 460 can have any suitable configuration, such as being the main lumen/cavity of the housing 404, a separately defined lumen integrated into the housing 404, or a separate tube extending through the housing 404. The vacuum lumen 460 is configured to be fluidly coupled to a vacuum source to pull gas or other fluid from the treatment location.

FIGS. 4G and 4H are simplified diagrams of the medical system 400 showing a control system 462 operably coupled to the medical device 402 with different configurations for the medical system 400. The control system 462 is coupled to the drive assembly 414 of the medical device 402 to control the deployment, expansion, and retraction of the elongate bands 406, as discussed above. For example, the control system 462 is configured to operate the drive assembly 414 to deploy and expand the elongate bands 406 at a treatment location, such as within a hollow sac. The control system 462 can control operation of a robotic motor carriage 430 (FIG. 4G) to drive movement of the carriage 418, or can operate the motor 424 (FIG. 4H) to rotate the leadscrew 416 to drive movement of the carriage 418.

During expansion of the bands 406, the control system 462 monitors operation of the drive assembly 414 to determine when the elongate bands 406 engage tissue. For example, the control system 462 can monitor a current draw of the drive assembly 414 to determine when the elongate bands engage tissue during expansion. When tissue is engaged, the current draw of the drive assembly 414 will increase due to the added resistance on the elongate bands 406 from the tissue. In some examples, the control system 462 can automatically stop operation of the drive assembly 414 in response to determining that the elongate bands 406 have engaged tissue. In other or additional examples, the control system 462 can notify a user with a suitable output 464, such as a display message, an audio message/alarm, a light, and so forth.

Further, the control system 462 can determine an expansion amount of the elongate bands 406 based on the operation of the drive assembly 414. For example, the control system 462 can correlate the amount of rotation of the leadscrew 416 to an amount of the looped intermediate portions 406b of the elongate bands 406 that is driven distally of the housing 404, the control system 462 can monitor the motor current, motor current rate of change, time, and motor speed, and/or sensors can be coupled within or to medical device 402 and in communication with the control system 462 to detect contact of the elongate bands 406 with tissue via pressure, strain, or conductivity data. With the expansion amount, the control system 462 also estimate a treatment area size (e.g., a size of an interior of the cyst) and determine one or more energy parameters associated with an energy treatment of the treatment area based on the expansion amount and treatment area size. In some examples, the energy parameters include a capacitance needed to treat the treatment area, the capacitance determined by the number of electrodes and treatment area size. For example, a greater capacitance is required for treatment of a relative larger area and/or for providing energy to a greater number of electrodes. In additional examples, the energy parameters can include one or more of voltage, a number of pulses, pulse length, dwell time between pulses, and/or sequence of pulses. These energy parameters can be adjusted based on the determined expansion amount.

To fully treat the interior tissue of a cyst in some embodiments, it may be desirable to have the electrodes 408, 410 utilize one position within the cyst for full treatment, or to have the electrodes 408, 410 be sequentially moved (e.g., rotated) to a series of different treatment positions within the cyst and have energy applied at each position until full coverage of the treatment area is achieved. Based on the treatment area size and a configuration of the electrodes 408, 410 (e.g., an energy treatment size from the electrodes 408, 410), the control system 462 can determine a rotation amount and number of rotations required for full treatment.

As shown in FIG. 4H, the medical system 400 further includes a roll drive 466 operably coupled to the housing 404. The roll drive 466 can be on-board with the medical device 402 or can be remote from the medical device, such as part of a backend mechanism, to roll the instrument 426 or other elongate device coupled to or manipulating/grasping the medical device 402. Accordingly, the control system 462 can operate the roll drive 466 to rotate the medical device 402 to a second location (e.g., a new alignment within the cyst). To avoid engagement with tissue during rotation, the control system 462 can operate the drive assembly 414 to partially retract the elongate bands 406 before rotation and operate the drive assembly 414 to expand the elongate bands 406 at the second treatment location.

In examples with the insufflation lumen 456, the control system 462 is configured to provide pressurized gas from an insufflation gas source 468 through the insufflation lumen 456 for insufflation at the treatment location. Insufflation can be utilized, for example, to push tissue away from the elongate bands 406 prior to and/or during partial or full retraction of the elongate bands 406 from the deployed configuration. Insufflation may also be utilized to inflate or otherwise expand the cavity of the treatment location.

In examples with the vacuum lumen 460, the control system 462 is configured to operate a vacuum source 470 to apply a vacuum through the vacuum lumen 460 for apposition at the treatment location. Apposition can be utilized, for example, to pull tissue against the electrodes 408, 410 prior to an energy treatment.

In examples with the support post 436 and the corresponding drive system 442, the control system 462 is configured to operate the drive system 442 to set an insertion depth for the elongate bands 406 with the support post 436 prior to operation of the drive assembly 414 for expansion of the elongate bands 406.

The medical system 400 further includes an energy source 472 (e.g., a power supply) that is electrically coupled to the control system 462 and the electrodes 408, 410 via suitable structure, including wires, traces, circuit boards, etc. The control system 462 is configured to control the application of bipolar energy from the energy source 472 to the electrodes 408, 410 for an energy treatment.

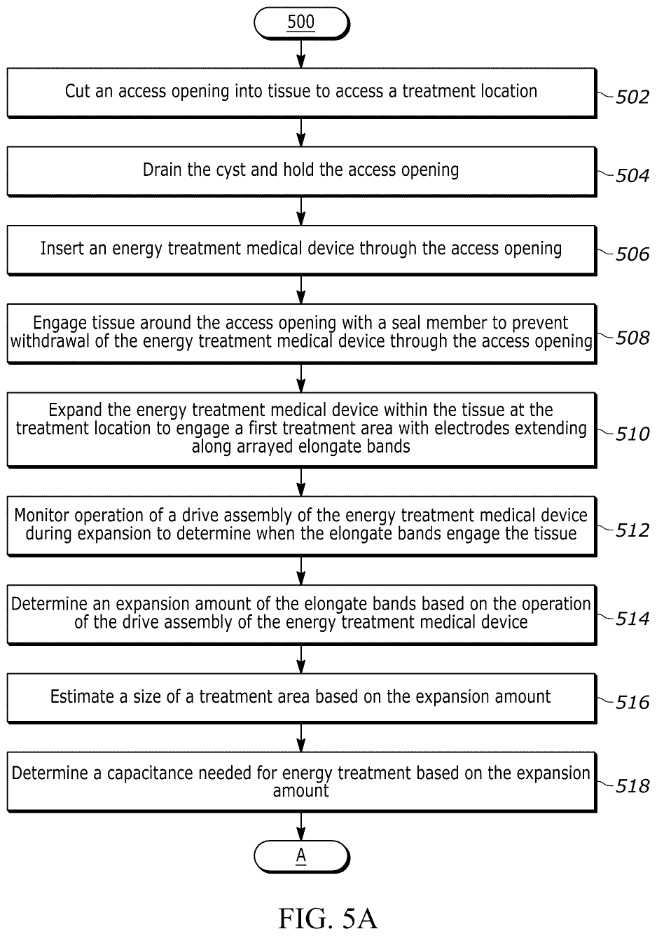

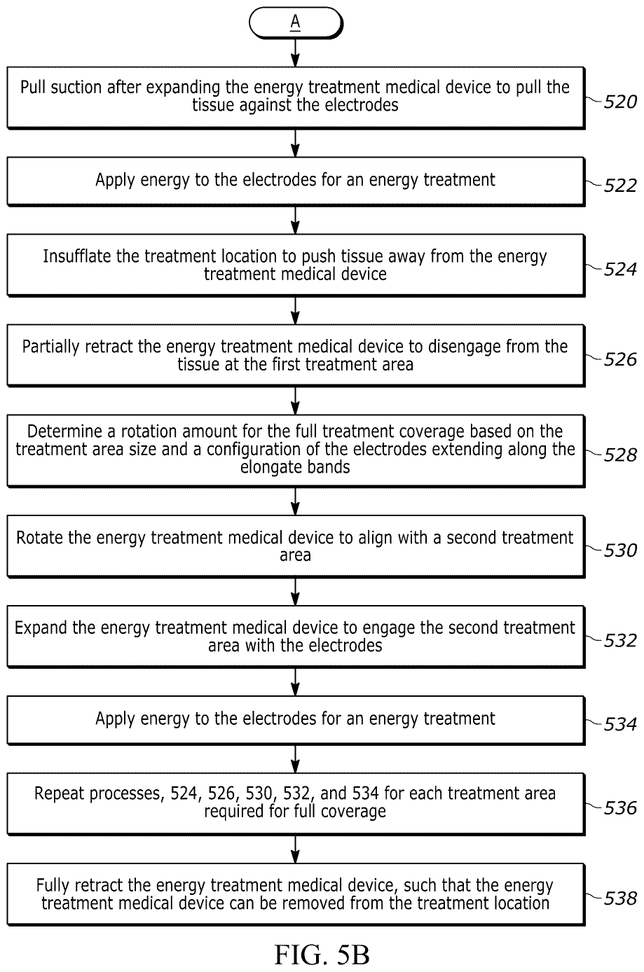

FIGS. 5A and 5B illustrates a method 500 for energy treatment according to some embodiments. The method 500 is illustrated as a set of operations or processes 502 through 538. Not all of the illustrated processes may be performed in all embodiments of the method 500. Additionally, one or more processes that are not expressly illustrated in FIGS. 5A and 5B may be included before, after, in between, or as part of the processes 502 through 536. Processes may also be performed in different orders. In some embodiments, one or more of the processes 502 through 538 may be implemented, at least in part, in the form of executable code stored on non-transitory, tangible, machine-readable media that when run by one or more processors (e.g., the processors of a controller) may cause the one or more processors to perform one or more of the processes. In one or more embodiments, the processes 502 through 538 may be performed by a controller.

In process 502, an access opening is cut into tissue to access a treatment location. In some examples, the treatment location includes fluid to be drained, such as a hollow sac or cyst. In these examples, in process 504, the method 500 includes draining the hollow sac or cyst and, if necessary, in process 506, holding the access opening open after draining the hollow sac or cyst.

In process 506, an energy treatment medical device (e.g., medical device 402) is inserted into the hollow sac or cyst (e.g., through the access opening) and, optionally tissue is engaged around the access opening with a seal member (e.g., seal member 458) after insertion of the energy treatment medical device to prevent withdrawal of the energy treatment medical device through the access opening.

In process 510, arrayed elongate bands (e.g., elongate bands 406) of the energy treatment medical device are expanded within the hollow sac or cyst to engage a first treatment area of an inner surface of the hollow sac or cyst with outer surfaces of the arrayed elongate bands. In some examples, expanding the arrayed elongate bands of the energy treatment medical device includes operating a motor (e.g. motor 424) to drive rotation of a leadscrew (e.g., leadscrew 416) within the energy treatment medical device to drive longitudinal translation of a carriage (e.g., carriage 418) rotatably coupled to the leadscrew, where ends of the arrayed elongate bands are coupled to the carriage. In some examples, expanding the arrayed elongate bands of the energy treatment medical device includes spacing portions of the arrayed elongate bands to define a treatment volume having a diameter of at least 50 mm, at least 70 mm, or at least 80 mm.

In process 512, operation of a drive assembly (e.g., drive assembly 414) of the energy treatment medical device is monitored during expansion to determine when the arrayed elongate bands engage the inner surface of the hollow sac or cyst. In some examples, the monitoring includes monitoring a current draw of the drive assembly. In process 514, an expansion amount of the arrayed elongate bands is determined based on operation of the drive assembly of the energy treatment medical device. In process 516, a size of a treatment area of the inner surface of the hollow sac or cyst is estimated based on the expansion amount and in process 518, one or more energy parameters associated with the energy treatment of the inner surface of the hollow sac or cyst is determined based on the expansion amount. In process 520, optionally, suction is pulled after expanding the energy treatment medical device to pull the tissue against the electrodes extending along the outer surfaces of the arrayed elongate bands and, in process 522, energy is applied to the electrodes for an energy treatment.

In process 524, optionally, the treatment location is insufflated to push tissue away from the energy treatment medical device and, in process 526, the energy treatment medical device is partially retracted to disengage from the tissue at the first treatment area. In some examples, process 524 and process 526 is performed simultaneously.

In process 528, a rotation amount for full treatment coverage of the inner surface of the hollow sac or cyst is determined based on the size of the treatment area and a configuration of the electrodes extending along the arrayed elongate bands and, in process 530, the energy treatment medical device is rotated to align with a second treatment area. In process 532, the energy treatment medical device is expanded to engage the second treatment area with the electrodes and, in process 534, energy is applied to the electrodes for an energy treatment.

In process 536, the method is repeated for each treatment area required for full coverage of the inner surface of the hollow sac or cyst. As such, process 536 includes partial retraction as in process 526 (and optionally process 524), rotation as in process 530, expansion as in process 532, and application of energy treatment as in process 534 in repetition to achieve full treatment coverage of the inner surface of the hollow sac or cyst. In process 538, the energy treatment medical device is fully retracted, such that the energy treatment medical device can be removed from the hollow sac or cyst.

One or more components of the embodiments discussed in this disclosure, such as control system 112, 462, may be implemented in software for execution on one or more processors of a computer system. The software may include code that when executed by the one or more processors, configures the one or more processors to perform various functionalities as discussed herein. The code may be stored in a non-transitory computer readable storage medium (e.g., a memory, magnetic storage, optical storage, solid-state storage, etc.). The computer readable storage medium may be part of a computer readable storage device, such as an electronic circuit, a semiconductor device, a semiconductor memory device, a read only memory (ROM), a flash memory, an erasable programmable read only memory (EPROM); a floppy diskette, a CD-ROM, an optical disk, a hard disk, or other storage device. The code may be downloaded via computer networks such as the Internet, Intranet, etc. for storage on the computer readable storage medium. The code may be executed by any of a wide variety of centralized or distributed data processing architectures. The programmed instructions of the code may be implemented as a number of separate programs or subroutines, or they may be integrated into a number of other aspects of the systems described herein. The components of the computing systems discussed herein may be connected using wired and/or wireless connections. In some examples, the wireless connections may use wireless communication protocols such as Bluetooth, near-field communication (NFC), Infrared Data Association (IrDA), home radio frequency (HomeRF), IEEE 802.11, Digital Enhanced Cordless Telecommunications (DECT), and wireless medical telemetry service (WMTS).

Various general-purpose computer systems may be used to perform one or more processes, methods, or functionalities described herein. Additionally or alternatively, various specialized computer systems may be used to perform one or more processes, methods, or functionalities described herein. In addition, a variety of programming languages may be used to implement one or more of the processes, methods, or functionalities described herein.

While certain embodiments and examples have been described above and shown in the accompanying drawings, it is to be understood that such embodiments and examples are merely illustrative and are not limited to the specific constructions and arrangements shown and described, since various other alternatives, modifications, and equivalents will be appreciated by those with ordinary skill in the art.

1. An energy treatment medical device comprising:
a housing;
an elongate band having ends received within the housing and a looped intermediate portion disposed distally of the housing; and
first and second electrodes spaced from one another along the elongate band, the first and second electrodes extending at least along the looped intermediate portion of the elongate band for energy treatment.

2. The energy treatment medical device of claim 1, wherein the first and second electrodes are disposed in side-by-side relation; and further comprising an insulator extending along the elongate band separating the first and second electrodes.

3. The energy treatment medical device of claim 1, wherein the second electrode comprises first and second portions disposed on either side of the first electrode; and further comprising insulators extending along the elongate band separating from the first electrode from the first and second portions of the second electrode.

4. The energy treatment medical device of claim 1, further comprising:
a leadscrew extending within the housing; and
a carriage rotatably coupled to the leadscrew and coupled to the housing, such that rotation of the leadscrew causes the carriage to translate longitudinally within the housing, the ends of the elongate band coupled to the carriage for deployment and retraction of the looped intermediate portion distally of the housing.

5. The energy treatment medical device of claim 4, further comprising:
a motor received at least partially within the housing, the motor coupled to the leadscrew to drive rotation thereof for deployment and retraction of the looped intermediate portion of the elongate band; or
an actuator interface comprising one or more capstans configured to be coupled to a backend mechanism, the one or more capstans coupled to the leadscrew to drive rotation thereof for deployment and retraction of the looped intermediate portion of the elongate band.

6. (canceled)

7. The energy treatment medical device of claim 1, wherein the elongate band comprises a first elongate band, and further comprising:
a second elongate band having ends received within the housing and a looped intermediate portion disposed distally of the housing; and
first and second electrodes spaced from one another along the second elongate band, the first and second electrodes extending at least along the looped intermediate portion of the second elongate band for energy treatment;
wherein the first and second elongate bands having an intersecting end point and the looped intermediate portions are radially arrayed about a longitudinal axis to define a treatment area.

8. The energy treatment medical device of claim 1, further comprising a support post coupled to the looped intermediate portion of the elongate band, the support post extending proximally from the looped intermediate portion of the elongate band into the housing and movable to allow deployment and retraction of the elongate band.

9. The energy treatment medical device of claim 8, further comprising a drive coupled to the support post, such that an insertion depth of the support post is adjustable to thereby adjust an amount of radial expansion of the elongate band.

10. The energy treatment medical device of claim 8, wherein the support post has a fixed insertion depth.

11. (canceled)

12. An energy treatment medical device comprising:
a housing;
a leadscrew received within the housing;
a carriage rotatably coupled to the leadscrew and coupled with the housing, such that rotation of the leadscrew drives longitudinal movement of the carriage; and
three elongate bands having electrodes extending longitudinally therealong, each elongate band having ends coupled to the carriage and a looped intermediate portion disposed distally of the housing, such that movement of the carriage causes expansion and retraction of the looped intermediate portions of the elongate bands.

13. The energy treatment medical device of claim 12, wherein the electrodes comprise first and second electrodes spaced from one another along the elongate band, the first and second electrodes extending at least along the looped intermediate portion of the elongate band for energy treatment.

14. (canceled)

15. The energy treatment medical device of claim 12, wherein the elongate bands are connected together at an intersection point, the elongate bands being radially arrayed to provide leaves defining a treatment area; and further comprising a support post coupled to the intersection point of the elongate bands, the support post extending proximally from the intersection point into the housing and being movable to allow deployment and retraction of the elongate bands.

16. (canceled)

17. The energy treatment medical device of claim 12, wherein the intermediate looped portions of the elongate bands are deployable from and retractable to a storage position within the housing such that a diameter of looped intermediate portions distal of the housing is equal to or less than a diameter of the housing.

18. The energy treatment medical device of claim 12, further comprising:
a motor at least partially received within the housing and coupled to the leadscrew to drive rotation of the leadscrew for deployment and retraction of the looped intermediate portions of the elongate bands; or
an actuator interface comprising one or more capstans configured to be coupled to a backend mechanism, the one or more capstans coupled to the leadscrew to drive rotation thereof for deployment and retraction of the looped intermediate portions of the elongate bands.

19-20. (canceled)

21. The energy treatment medical device of claim 12, wherein the housing defines an insufflation lumen configured to be coupled to an insufflation gas source to provide pressurized gas for insufflation at a treatment location; and
further comprising a seal member extending around the housing, the seal member configured to engage tissue at an insertion opening to the treatment location.

22. (canceled)

23. The energy treatment medical device of claim 12, wherein the housing defines a vacuum lumen configured to be coupled to a vacuum source for apposition at the treatment location prior to energy treatment.

24. (canceled)

25. An energy treatment medical device comprising:
a housing;
a drive assembly;
a carriage coupled to the drive assembly, operation of the drive assembly configured to drive longitudinal movement of the carriage;
three elongate bands having electrodes extending longitudinally therealong, each elongate band having ends coupled to the carriage and a looped intermediate portion disposed distally of the housing, such that movement of the carriage causes expansion and retraction of the looped intermediate portions of the elongate bands;
a support shaft coupled to the drive assembly, the looped intermediate portions of the elongate bands coupled to an end of the support shaft, operational of the drive assembly configured to drive longitudinal movement of the support shaft to set an insertion depth for the elongate bands.

26-27. (canceled)

28. The energy treatment medical device of claim 25, wherein the drive assembly comprises:
a first drive configured to drive longitudinal movement of the carriage; and
a second drive configured to drive longitudinal movement of the support shaft.

29. The energy treatment medical device of claim 28, wherein:
the first drive comprises a first leadscrew received within the housing, and the carriage is rotatably coupled to the first leadscrew and coupled to the housing, such that rotation of the first leadscrew drives longitudinal movement of the carriage; and
the second drive comprises a second leadscrew received within the housing, and the support shaft is rotatably coupled to the second leadscrew and coupled to the housing, such that rotation of the second leadscrew drives longitudinal movement of the support shaft.

30-32. (canceled)

33. The energy treatment medical device of claim 25, wherein the housing defines:
an insufflation lumen configured to be coupled to an insufflation gas source to provide pressurized gas for insufflation at a treatment location; and
a vacuum lumen configured to be coupled to a vacuum source for apposition at a treatment location prior to energy treatment.

34-71. (canceled)
